(19) United States
(12) Patent Application Publication
(10) Pub. No.: US 2024/0350929 A1
(43) Pub. Date: Oct. 24, 2024

(54) CHEST PAIN REDUCTION SYSTEM

(71) Applicants: Anthony Sparrow, Delphos, OH (US); Donna Derner, Delphos, OH (US)

(72) Inventors: Anthony Sparrow, Delphos, OH (US); Donna Derner, Delphos, OH (US)

(21) Appl. No.: 18/136,586

(22) Filed: Apr. 19, 2023

(51) Int. Cl. A63H 3/00; A61F 5/03; A63B 23/18; A63H 3/02

(52) U.S. Cl. CPC A63H 3/003; A61F 5/03; A63B 23/18; A63H 3/02

(57) ABSTRACT

A chest pain reduction system for holding a teddy bear against a post-operated chest to squeeze such that the teddy bear reduces pain when coughing includes a post-operative brace facilitating performing respiratory exercises by hugging the post-operative brace against a chest of a user such that the post-operative brace supports the chest when performing respiratory exercises. The post-operative brace comprises a teddy bear. The teddy bear is weighted and firmed by a weighted member to define a bear brace. The bear brace includes a pair of legs, a pair of arms, and a head. A harness is worn on a torso of a user. The post-operative brace is couplable to the harness, wherein the harness holds the post-operative brace against the chest of the user when the harness is worn on the torso of the user such that the user hugs the post-operative brace when performing respiratory exercises.

### CROSS-REFERENCE TO RELATED APPLICATIONS

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to teddy bears and more particularly pertains to a new teddy bear for holding a teddy bear against a post-operated chest to squeeze such that the teddy bear reduces pain when coughing.

### (2) Description of Related Art including information disclosed under 37 CFR 1.97 and 1.98

The prior art relates to teddy bears and includes a variety of teddy bears having tethering members for attaching to a user. Known prior art does not include a teddy bear being firm and weighted having tethering members to secure to a post-operated chest of the user such that the user may hug the teddy bear when coughing to reduce chest pain.

### BRIEF SUMMARY OF THE INVENTION

An embodiment of the disclosure meets the needs presented above by generally comprising a post-operative brace configured to facilitate performing respiratory exercises by hugging the post-operative brace against a chest of a user such that the post-operative brace supports the chest when performing respiratory exercises. The post-operative brace comprises a teddy bear. The teddy bear is weighted and firmed by a weighted member to define a bear brace. The bear brace includes a pair of legs, a pair of arms, and a head. A harness is configured to be worn on a torso of a user. The post-operative brace is couplable to the harness, wherein the harness is configured to hold the post-operative brace against the chest of the user when the harness is worn on the torso of the user such that the user hugs the post-operative brace when performing respiratory exercises.

Another embodiment of the disclosure comprises a method including the steps of fastening a harness to a torso of a user. The harness includes a brace holder, a pair of tethers each extending from the brace holder, and a fastener securing the tethers to the brace holder. The holder is positioned against a chest of the user. A post-operative brace positions within the brace holder of the harness. The post-operative brace comprises a teddy bear to define a bear brace. The user squeezes the bear brace against the chest when performing respiratory exercises to alleviate pain.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a chest pain reduction system according to an embodiment of the disclosure.

FIG. 2 is a back view of an embodiment of the disclosure.

FIG. 3 is a top perspective view of an embodiment of the disclosure.

FIG. 4 is a front detail view of an embodiment of the disclosure.

FIG. 5 is a front in-use view of an embodiment of the disclosure.

FIG. 6 is a cross-sectional view of an embodiment of the disclosure taken along Line 6-6 of FIG. 5.

### DETAILED DESCRIPTION OF THE INVENTION

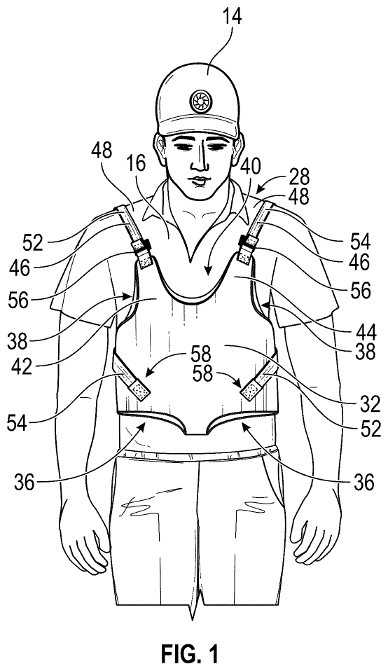

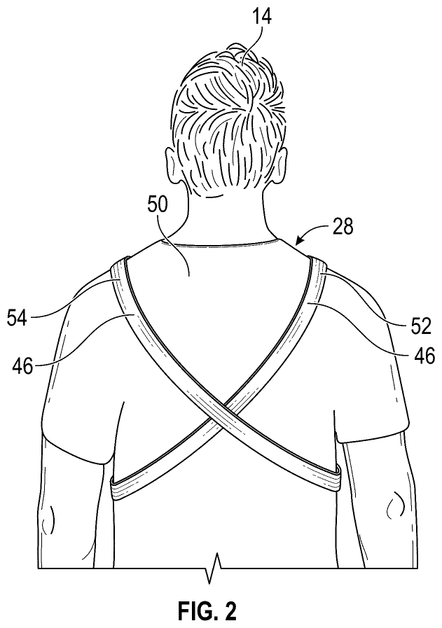

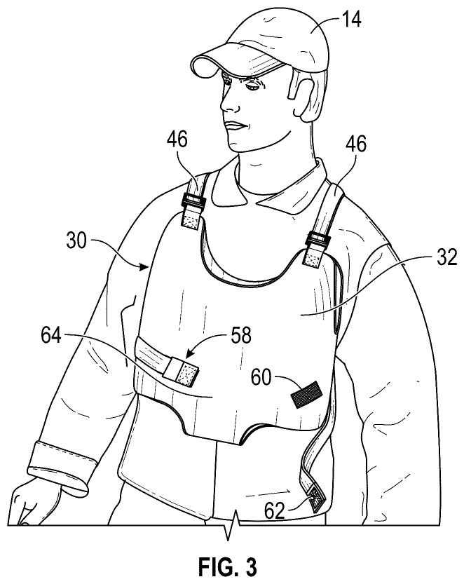

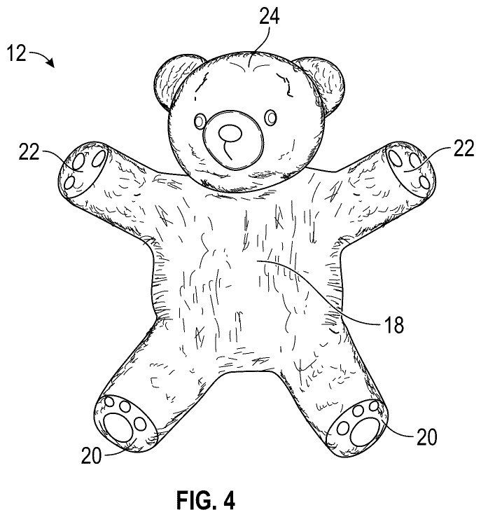

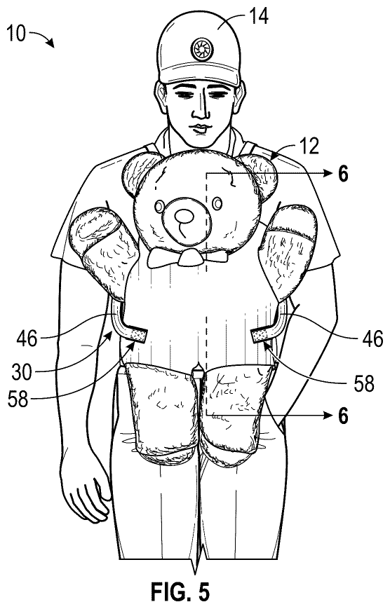

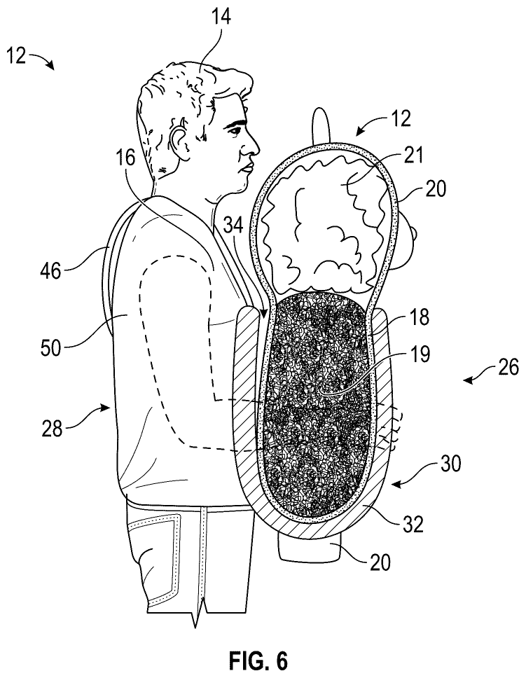

With reference now to the drawings, and in particular to FIGS. 1 through 6 thereof, a new teddy bear embodying the principles and concepts of an embodiment of the disclosure and generally designated by the reference numeral 10 will be described.

As best illustrated in FIGS. 1 through 6, the chest pain reduction system 10 generally comprises a post-operative brace 12 configured to facilitate performing respiratory exercises by hugging the post-operative brace 12 against a chest 16 of a user 14 such that the post-operative brace 12 supports the chest 16 when performing respiratory exercises. The post-operative brace 12 comprises a teddy bear. The teddy bear is weighted and firmed by a weighted member 19 positioned within the teddy bear to define a bear brace 18. The weighted member 19 may comprise a bag of sand positioned between stuffing 21 within the bear brace 18. The bear brace 18 includes a pair of legs 20, a pair of arms 22, and a head 24. The bear brace 18 may have a coat of synthetic fur, or any other material typically used to create a conventional teddy bear.

A harness 26 is configured to be worn on a torso 28 of a user 14. The post-operative brace 12 is couplable to the harness 26, wherein the harness 26 is configured to hold the post-operative brace 12 against the chest 16 of the user 14 when the harness 26 is worn on the torso 28 of the user 14 such that the user 14 hugs the post-operative brace 12 when performing respiratory exercises. The harness 26 includes a brace holder 30 removably holding the post-operative brace 12. The brace holder 30 comprises a covering 32 enclosing a receiving space 34. A bottom side of the covering 32 has a pair of leg openings 36 extending therethrough and into the receiving space 34. A top side of the covering 32 has a pair of arm openings 38 extending therethrough and into the receiving space 34.

Furthermore, the top side of the covering 32 has a head opening 40 extending therethrough and into the receiving space 34. The head opening 40 is positioned between the pair of arm openings 38 to form a first strap 42 and a second strap 44 of the covering 32. The receiving space 34 receives the bear brace 18, wherein the head 24 of the bear brace 18 is positioned within the head opening 40, each of the arms 22 of the bear brace 18 are positioned within a respective one of the arm openings 38, and each of the legs 20 of the bear brace 18 are positioned within a respective one of the leg openings 36. The first 42 and second 44 straps may be selectively moveable from each other to expand the head opening 40, wherein the bear brace 18 is insertable through the expanded head opening 40.

The harness 26 includes a pair of tethers 46 configured to extend over a pair of shoulders 48 of the user 14 and crisscross on a back 50 of the torso 28 of the user 14. The pair of tethers 46 include a first tether 52 and a second tether 54. The first tether 52 is coupled to and extends outwardly from the first strap 42 of the covering 32, and the second tether 54 is coupled to and extends outwardly from the second strap 44 of the covering 32. Each of the tethers 46 have a respective one of a pair of adjustment members 56, wherein the first tether 52 and the second tether 54 each have an adjustable length. The adjustable length of each of the tethers 46 may be increased or decreased when securing the harness 26 to the torso 28 of the user 14.

A fastener 58 configured to fasten the tethers 46 to the user 14 includes a pair of first fastening members 60 and a pair of second fastening members 62. Each of the second fastening members 62 is engageable with one of the pair of first fastening members 60. The first fastening members 60 are positioned on a front side 64 of the covering 32. Each of the second fastening members 62 are positioned on an end of a respective one of the pair of tethers 46, wherein an end of each of the tethers 46 fastens with the front side 64 of the covering 32. The fastener 58 is a hook and loop fastener 58, wherein each of the first fastening members 60 comprises a loop material and each of the second fastening members 62 comprises a hook material. Alternatively, the fastener 58 may be a snap button fastener 58, wherein each of the first fastening member comprises a socket and each of the second fastening members 62 comprises a stud.

In use, the harness 26 fastens to the torso 28 of the user 14. Each of the tethers 46 positions upon one of the pair of shoulders 48 of the user 14. The first tether 52 is positioned upon a first shoulder of the pair of shoulders 48 and the second tether 54 is positioned upon a second shoulder of said the of shoulders 48. The pair of tethers 46 crisscrosses by crossing the first tether 52 over the second tether 54. The tethers 46 fasten to the holder with the fastener 58. The first tether 52 extends underneath the second shoulder to the front side 64 of the holder and the second tether 54 extends underneath the first shoulder to the front side 64 of the holder. The post-operative brace 12 positions within the brace holder 30 of the harness 26. The user 14 squeezes the post-operative brace 12 against the chest 16 when performing respiratory exercises to alleviate pain.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A chest pain reduction system configured to alleviate chest pain, said chest pain reduction system comprising:
a post-operative brace configured to facilitate performing respiratory exercises by hugging the post-operative brace against a chest of a user such that the post-operative brace supports the chest when performing respiratory exercises, said post-operative brace comprising a teddy bear, said teddy bear being weighted and firmed by a weighted member to define a bear brace, said bear brace including a pair of legs, a pair of arms, and a head; and
a harness being configured to be worn on a torso of a user, said post-operative brace being couplable to said harness, wherein said harness is configured to hold said post-operative brace against the chest of the user when said harness is worn on the torso of the user such that the user hugs the post-operative brace when performing respiratory exercises.

2. The chest pain reduction system of claim 1, wherein said harness includes a brace holder removably holding said post-operative brace, said brace holder comprising a covering enclosing a receiving space, a bottom side of said covering having a pair of leg openings extending therethrough and into said receiving space, a top side of said covering having a pair of arm openings extending therethrough and into said receiving space, said top side of said covering having a head opening extending therethrough and into said receiving space, said head opening being positioned between said pair of arm openings to form a first strap and a second strap of said covering, said receiving space receiving said bear brace, wherein said head of said bear brace is positioned within said head opening, each of said arms of said bear brace are positioned within a respective one of said arm openings, and each of said legs of said bear brace are positioned within a respective one of said leg openings, said first and second straps being selectively movable away from each other to expand said head opening, wherein said bear brace is insertable through said head opening.

3. The chest pain reduction system of claim 2, further including a pair of tethers configured to extend over a pair of shoulders of the user and crisscross on a back of the torso of the user, said pair of tethers including a first tether and a second tether, said first tether being coupled to and extending outwardly from said first strap of said covering, said second tether being coupled to and extending outwardly from said second strap of said covering, each of said tethers having one of a pair of adjustment members, wherein said first tether and said second tether each have an adjustable length.

4. The chest pain reduction system of claim 3, further including a fastener configured to fasten said tethers to the user, said fastener including a pair of first fastening members and a pair of second fastening members, each of said second fastening members being engageable with one of the pair of first fastening members, said first fastening members being positioned on a front side of said covering, each of said second fastening members being positioned on an end of a respective one of said pair of tethers, wherein an end of each of said tethers fastens with said front side of said covering.

5. A chest pain reduction system configured to alleviate chest pain, said chest pain reduction system comprising:
a post-operative brace configured to facilitate performing respiratory exercises by hugging the post-operative brace against a chest of a user such that the post-operative brace supports the chest when performing respiratory exercises, said post-operative brace comprising a teddy bear, said teddy bear being weighted and firmed by a weighted member to define a bear brace, said bear brace including a pair of legs, a pair of arms, and a head;
a harness being configured to be worn on a torso of a user, said post-operative brace being couplable to said harness, wherein said harness is configured to hold said post-operative brace against the chest of the user when said harness is worn on the torso of the user such that the user hugs the post-operative brace when performing respiratory exercises, said harness including:
a brace holder removably holding said post-operative brace, said brace holder comprising a covering enclosing a receiving space, a bottom side of said covering having a pair of leg openings extending therethrough and into said receiving space, a top side of said covering having a pair of arm openings extending therethrough and into said receiving space, said top side of said covering having a head opening extending therethrough and into said receiving space, said head opening being positioned between said pair of arm openings to form a first strap and a second strap of said covering, said receiving space receiving said bear brace, wherein said head of said bear brace is positioned within said head opening, each of said arms of said bear brace are positioned within a respective one of said arm openings, and each of said legs of said bear brace are positioned within a respective one of said leg openings, said first and second straps being selectively movable away from each other to expand said head opening, wherein said bear brace is insertable through said head opening;
a pair of tethers configured to extend over a pair of shoulders of the user and crisscross on a back of the torso of the user, said pair of tethers including a first tether and a second tether, said first tether being coupled to and extending outwardly from said first strap of said covering, said second tether being coupled to and extending outwardly from said second strap of said covering, each of said tethers having one of a pair of adjustment members, wherein said first tether and said second tether each have an adjustable length; and
a fastener configured to fasten said tethers to the user, said fastener including a pair of first fastening members and a pair of second fastening members, each of said second fastening members being engageable with one of the pair of first fastening members, said first fastening members being positioned on a front side of said covering, each of said second fastening members being positioned on an end of a respective one of said pair of tethers, wherein an end of each of said tethers fastens with said front side of said covering.

6. A method for alleviating pain when performing respiratory exercises including the steps of:
fastening a harness to a torso of a user, said harness including a brace holder, a pair of tethers each extending from said brace holder, and a fastener securing said tethers to said brace holder, said holder being positioned against a chest of the user;
positioning a post-operative brace within said brace holder of said harness, said post-operative brace comprising a teddy bear to define a bear brace;
squeezing said bear brace against the chest of the user when performing respiratory exercises to alleviate pain.

7. The method of claim 7, wherein the step of fastening a harness to a user further includes the steps of:
positioning each of said tethers upon one of a pair of shoulders of the user, said pair of tethers including a first tether and a second tether, said first tether being positioned upon a first shoulder of said pair of shoulders, said second tether being positioned upon a second shoulder of said pair of shoulders;
crisscrossing said pair of tethers by crossing said first tether over said second tether; and
fastening said tethers to said holder with said fastener, said first tether extending underneath said second shoulder to a front side of said holder, said second tether extending underneath said first shoulder to said front side of said holder.
